(19) United States
(12) Patent Application Publication
(10) Pub. No.: US 2006/0253034 A1
(43) Pub. Date: Nov. 9, 2006

(54) Ultrasonic diagnostic system

(75) Inventor: Hiroshi Fukukita, Tokyo (JP)

Correspondence Address: HAMRE, SCHUMANN, MUELLER & LARSON P.C., P.O. BOX 2902-0902, MINNEAPOLIS, MN, 55402, US

(73) Assignee: MATSUSHITA ELECTRIC INDUSTRIAL CO., ., Osaka (JP)

(21) Appl. No.: 10/569,014

(22) PCT Filed: Mar. 8, 2004
(86) PCT No.: PCT/JP04/02986

§ 371 Date: Feb. 21, 2006

(30) Foreign Application Priority Data
Sep. 26, 2003 (JP) 2003-336108

(51) Int. Cl. A61B 8/14

(52) U.S. Cl. 600/472

(57) ABSTRACT

An ultrasonic diagnostic system capable of phasing a received signal from an electroacoustic transducer arranged two-dimensionally with high precision is provided. The ultrasonic diagnostic system includes a sub-beam former (16) having amplifying sections (8, 9) for amplifying received signals of vibrators (1, 2), variable amplitude sections (10 to 13) for controlling amplitudes of an inverted output signal and a non-inverted output signal from the amplifying sections, a fixed-delay section (14) for imparting a delay time of a quarter of one period of the received signal to an added signal of the variable amplitude section, and an additing section (15) for adding the added signal of the variable amplitude section to an output signal of the fixed delay section, and a sub-beam former (17) having the same configuration as that of the sub-beam former (16), with respect to the received signals of the vibrators (3, 4), wherein the output signals from the sub-beam formers (16, 17) are subjected to delay addition by a main beam former (18).

### TECHNICAL FIELD

The present invention relates to an ultrasonic diagnostic system that has a two-dimensional array in which vibrators are arranged, and scans a subject three-dimensionally.

### BACKGROUND ART

As shown in FIG. 7, a conventional ultrasonic diagnostic system includes a two-dimensional array 107 in which a sub-array 105 composed of vibrators 101, 102, and a sub-array 106 composed of vibrators 103, 104 are arranged two-dimensionally. Received signals from the vibrators 101, 102 constituting the sub-array 105 respectively are input to amplifying sections 108, 109, and the amplifying sections 108, 109 output a non-inverted output signal (+) and an inverted output signal (−). The non-inverted output signal (+) and the inverted output signal (−) from the amplifying section 108 respectively are supplied to variable amplitude sections 110, 111 via a cross point switch 181, and output signals thereof are added to be input to a +45-degree phase shifter 114.

Furthermore, the non-inverted output signal (+) and the inverted output signal (−) from the amplifying section 109 respectively are supplied to the variable amplitude sections 112, 113 via a cross point switch 191, and output signals thereof are added to be input to a −45-degree phase shifter 115.

The output signals from the +45-degree phase shifter 114 and the −45-degree phase shifter 115 are added to be input to a main beam former 118. Herein, the amplifying sections 108, 109, the cross point switches 181, 191, the variable amplitude sections 110, 111, 112, 113, the +45-degree phase shifter 114, and the −45-degree phase shifter 115 constitute a sub-beam former 116.

Furthermore, received signals from the vibrators 103, 104 constituting the sub-array 106 are input to a sub-beam former 117. The internal configuration of the sub-beam former 117 is the same as that of the sub-beam former 116. The signals from the sub-beam formers 116 and 117 are subjected to delay addition by the main beam former, processed to be converted to an image signal by a signal processing section 119, and displayed on a display section 120.

In the above-mentioned sub-beam former configuration, the amplitudes of the received signals are controlled by the cross point switches 181, 191 and the variable amplitude sections 110 to 113, whereby the phases of the received signals are controlled, and the received signals from the vibrators in the sub-array are phased (e.g., see U.S. Pat. No. 6,013,032 (col. 8-10, FIGS. 6, 7, and 9).

However, in the conventional ultrasonic diagnostic system, there is the following problem: in order to shift the phase of a received signal, ±45-degree (±π/4) phase shifters in two channels are used, which makes it difficult to adjust a phase with good precision.

### DISCLOSURE OF INVENTION

The present invention has been achieved in order to solve the conventional problem, and its object is to provide an ultrasonic diagnostic system capable of phasing a received signal with good precision.

In order to achieve the above object, a first ultrasonic diagnostic system according to the present invention includes: electroacoustic conversion means in which a plurality of sub-arrays each composed of a plurality of electroacoustic transducers are arranged at least two-dimensionally; a sub-beam former that is provided on the sub-array basis, generates signals having different polarities with respect to a received signal from the electroacoustic transducer in the sub-array, obtains a first signal and a second signal by controlling amplitudes of the signals having different polarities of each electroacoustic transducer in the sub-array, followed by adding, imparts a delay time difference corresponding to a quarter of one period of the received signal between the first signal and the second signal by delay means provided inside, and adds the first signal and the second signal to which the delay time difference is imparted; and a main beam former for subjecting a signal output from the sub-beam former to delay addition.

According to the above configuration, a received signal can be phased with high precision.

Furthermore, in the first ultrasonic diagnostic system according to the present invention, the delay means is capable of switching the delay time difference between a quarter of one period of a fundamental of the received signal and a quarter of one period of a harmonic of the received signal.

According to the above configuration, the display of a fundamental video and the display of a harmonic video can be switched.

Furthermore, in the first ultrasonic diagnostic system according to the present invention, the delay means imparts a delay time corresponding to a quarter of one period of the received signal to one of the first signal and the second signal.

According to the above configuration, a received signal can be phased with high precision.

Furthermore, in order to achieve the above-mentioned object, a second ultrasonic diagnostic system according to the present invention includes: electroacoustic conversion means in which a plurality of sub-arrays each composed of a plurality of electroacoustic transducers are arranged at least two-dimensionally; a sub-beam former that is provided on the sub-array basis, generates signals having different polarities with respect to a received signal from the electroacoustic transducer in the sub-array, obtains a first signal and a second signal by controlling amplitudes of the signals having different polarities of each electroacoustic transducer in the sub-array, followed by adding, imparts a predetermined phase shift amount to one of the first signal and the second signal by phase shift means provided inside, and adds the first signal or the second signal to which the predetermined phase shift amount is imparted to each other; and a main beam former for subjecting a signal output from the sub-beam former to delay addition.

According to the above configuration, a received signal can be phased with high precision.

Furthermore, in the second ultrasonic diagnostic system according to the present invention, the phase shift means is composed of phase shift circuits in two stages, each having a phase shift amount of 45 degrees, and each of the phase shift circuits in two stages is configured so as to include a capacitor and a resistor.

According to the above configuration, a received signal can be phased with high precision.

Furthermore, in order to achieve the above-mentioned object, a third ultrasonic diagnostic system according to the present invention includes: electroacoustic conversion means in which a plurality of sub-arrays each composed of a plurality of electroacoustic transducers are arranged at least two-dimensionally; parallel adding means that is provided on the sub-array basis, generates signals having different polarities with respect to a received signal from the electroacoustic transducer in the sub-array, and obtains a first signal and a second signal by controlling amplitudes of the signals having different polarities of each electroacoustic transducer in the sub-array, followed by adding; a first main beam former for subjecting a first signal added by the parallel adding means to delay addition; a second main beam former for subjecting a second signal added by the parallel adding means to delay addition; delay means for imparting a delay time difference corresponding to a quarter of one period of the received signal between an output signal of the first main beam former and an output signal of the second main beam former; and adding means for adding an output signal of the first main beam former and an output signal of the second main beam former, to which the delay time difference is imparted by the delay means.

According to the above configuration, a received signal can be phased with high precision.

Furthermore, in order to achieve the above object, a fourth ultrasonic diagnostic system according to the present invention includes: electroacoustic conversion means in which a plurality of sub-arrays, each composed of a plurality of electroacoustic transducers, are arranged at least two-dimensionally; parallel adding means that is provided on the sub-array basis, generates signals having different polarities with respect to a received signal from the electroacoustic transducer in the sub-array, and obtains a first signal and a second signal by controlling amplitudes of the signals having different polarities of each electroacoustic transducer in the sub-array, followed by adding; a first main beam former for subjecting a first signal added by the parallel adding means to delay addition; a second main beam former for subjecting a second signal added by the parallel adding means to delay addition; phase shift means for imparting a phase difference of 90 degrees between an output signal of the first main beam former and an output signal of the second main beam former; and adding means for adding an output signal of the first main beam former and an output signal of the second main beam former, to which the phase difference of 90 degrees is imparted by the phase shift means.

According to the above configuration, a received signal can be phased with high precision.

According to the present invention, the following special effect is exhibited: an ultrasonic diagnostic system capable of phasing a received signal from an electroacoustic transducer arranged two dimensionally with high precision can be provided.

### BRIEF DESCRIPTION OF DRAWINGS

FIG. 1A is a block diagram showing one exemplary configuration of a receiving section in an ultrasonic diagnostic system according to a first embodiment of the present invention.

FIG. 1B is a schematic diagram showing an exemplary configuration of a two-dimensional array composed of a number of vibrators including vibrators 1 to 4 in FIG. 1A.

FIG. 2 is a block diagram showing an exemplary internal configuration of a sub-beam former of a receiving section in an ultrasonic diagnostic system according to a second embodiment of the present invention.

FIG. 3 is a block diagram showing an exemplary internal configuration of a sub-beam former of a receiving section in an ultrasonic diagnostic system according to a third embodiment of the present invention.

FIG. 4 is a detailed block diagram showing an exemplary internal configuration of a phase shifter shown in FIG. 3.

FIG. 5 is a block diagram showing an exemplary configuration of a receiving section in an ultrasonic diagnostic system according to a fourth embodiment of the present invention.

FIG. 6 is a block diagram showing a modified example of the receiving section in the ultrasonic diagnostic system according to the fourth embodiment of the present invention.

FIG. 7 is a block diagram showing an exemplary configuration of a conventional ultrasonic diagnostic system.

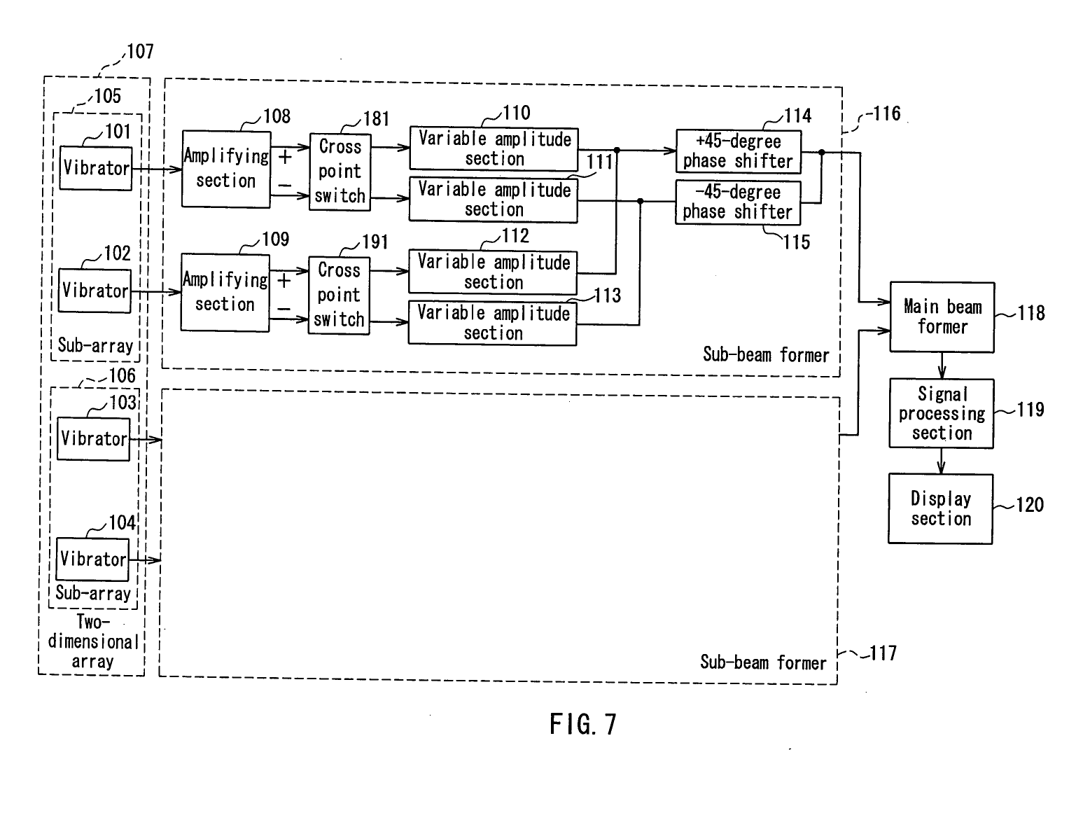

### DESCRIPTION OF THE INVENTION

Hereinafter, preferable embodiments of the present invention will be described with reference to the drawings.

### First Embodiment

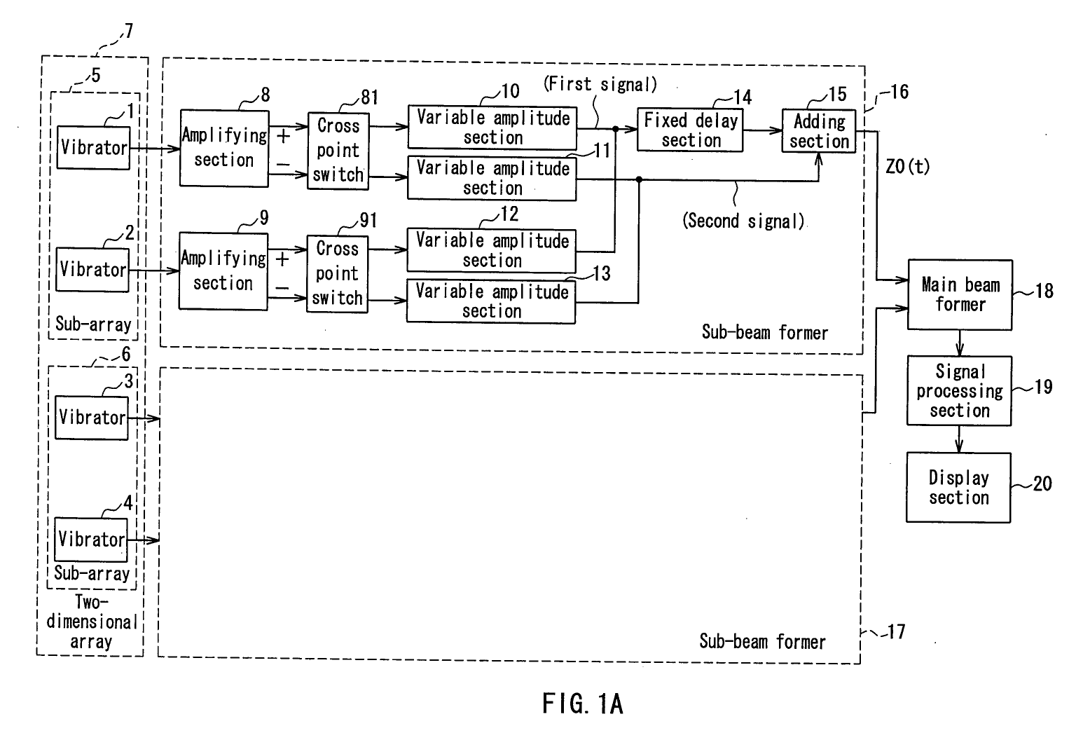

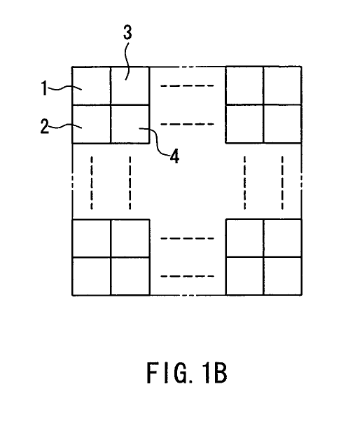

FIG. 1A is a block diagram showing one exemplary configuration of a receiving section in an ultrasonic diagnostic system according to a first embodiment of the present invention.

In FIG. 1A, vibrators 1 to 4 are composed of electroacoustic transducers, and convert an acoustic echo signal to a received signal. The vibrators 1 and 2 constitute a sub-array 5, the vibrators 3 and 4 constitute a sub-array 6, and the sub-arrays 5 and 6 constitute a two-dimensional array 7. Although only the vibrators 1 to 4 are illustrated in FIG. 1A, actually, a number of vibrators are arranged two-dimensionally as shown in FIG. 1B.

Amplifying sections 8, 9 respectively output a non-inverted output signal (+) and an inverted output signal (−) of the received signals from the vibrators 1, 2. Variable amplitude sections 10, 11 are connected to the amplifying section 8 via a cross point switch 81, and variable amplitude sections 12, 13 are connected to the amplifying section 9 via a cross point switch 91. The output signals of the variable amplitude sections 10, 12 are added, and the added signal (first signal) is supplied to a fixed delay section 14. Furthermore, the output signals of the variable amplitude sections 11, 13 are added, and the added signal (second signal) is added to the output signal of the fixed delay section 14 in an adding section 15. The amplifying sections 8, 9, the cross point switches 81, 91, the variable amplitude sections 10, 11, 12, 13, the fixed delay section 14, and the adding section 15 constitute a sub-beam former 16.

Furthermore, the received signals from the vibrators 3, 4 are input to a sub-beam former 17. The internal configuration of the sub-beam former 17 is the same as that of the sub-beam former 16.

The output signals of the sub-beam formers 16, 17 are subjected to delay addition by a main beam former 18. The output signal of the main beam former 18 is processed to be an image signal by a signal processing section 19. The image signal from the signal processing section 19 is displayed on a display section 20.

Next, an operation of an ultrasonic diagnostic system configured as described above will be described.

First, the vibrator 1 generates a received signal a(t)cos(2π·f1·t). Herein, t is a time, a(t) is an envelope of the received signal, and f1 is a center frequency of the received signal. The amplifying section 8 outputs a non-inverted output signal a(t)cos(2π·f1·t), and an inverted output signal −a(t)cos(2π·f1·t). Depending upon the connection state between a non-inverted output and an inverted output in the cross point switch 81, the variable amplitude section 10 multiplies the non-inverted output signal or the inverted output signal by a coefficient w(0) to output ±w(0)·a(t)cos(2π·f1·t). Furthermore, depending upon the connection state between the non-inverted output and the inverted output in the cross point switch 81, the variable amplitude section 11 multiplies the non-inverted output signal or the inverted output signal by a coefficient w(1) to output X1(t)=±w(1)·a(t)cos(2π·f1·t). The fixed delay section 14 imparts a delay time ΔT=T1/4, which is a quarter of one period T1=1/f of the received signal to the output signal of the variable amplitude section 10, and generates an output signal X0(t) represented by the following expression depending upon the connection state of the cross point switch 81.
X0(t)=±w(0)·a(t−ΔT)cos(2π·f1(t−ΔT)) (1)

As the fixed delay section 14, components such as a charge-coupled device and a sample-and-hold circuit capable of variably controlling a delay time with high precision with a clock are desired. Assuming that 2π·f1·ΔT=π/2 and a(t−ΔT) are approximated to a(t), Expression (1) can be represented as follows.
X0(t)=±w(0)·a(t)cos(2·f1·t−π/2) (2)

An output signal X0(t) of the fixed delay section 14 is added to the output signal X1(t) of the variable amplitude section 11 in the adding section 15 to be a sub-beam former output signal Z0(t). For example, in the case where w(0)=0, w(1)=1, and the non-inverted output of the amplifying section 8 is connected to the variable amplitude section 11, the sub-beam former output signal is represented by the following expression.
Z0(t)≈a(t)cos(2π·f1·t) (3)

Furthermore, in the case where w(0)=0.71, w(1)=0.71, the non-inverted output of the amplifying section 8 is connected to the variable amplitude section 10, and the non-inverted output of the amplifying section 8 is connected to the variable amplitude section 11, the sub-beam former output signal is represented by the following expression.
Z0(t)≈a(t)cos(2π·f1·t−π/4) (4)

Furthermore, in the case where w(0)=1, w(1)=0, and the non-inverted output of the amplifying section 8 is connected to the variable amplitude section 10, the sub-beam former output signal is represented by the following expression.
Z0(t)≈a(t)cos(2π·f1·t−π/2) (5)

Furthermore, in the case where w(0)=0.71, w(1)=0.71, the non-inverted output of the amplifying section 8 is connected to the variable amplitude section 10, and the inverted output of the amplifying section 8 is connected to the variable amplitude section 11, the sub-beam former output signal is represented by the following expression.
Z0(t)≈a(t)cos(2π·f1·t−3π/4) (6)

Furthermore, in the case where w(0)=0, w(1)=1, and the inverted output of the amplifying section 8 is connected to the variable amplitude section 11, the sub-beam former output signal is represented by the following expression.
Z0(t)≈a(t)cos(2π·f1·t−π) (7)

Furthermore, in the case where w(0)=0.71, w(1)=0.71, the inverted output of the amplifying section 8 is connected to the variable amplitude section 10, and the inverted output of the amplifying section 8 is connected to the variable amplitude section 11, the sub-beam former output signal is represented by the following expression.
Z0(t)≈a(t)cos(2π·f1·t−5π/4) (8)

Furthermore, in the case where w(0)=1, w(1)=0, and the inverted output of the amplifying section 8 is connected to the variable amplitude section 10, the sub-beam former output signal is represented by the following expression:
Z0(t)≈a(t)cos(2π·f1·t−3π/2) (9)

Furthermore, in the case where w(0)=0.71, w(1)=0.71, the inverted output of the amplifying section 8 is connected to the variable amplitude section 10, and the non-inverted output of the amplifying section 8 is connected to the variable amplitude section 11, the sub-beam former output signal is represented by the following expression.
Z0(t)≈a(t)cos(2π·f1·t−7π/4) (10)

Thus, a phase φa of the received signal a(t)cos(2π·f1·t) of the vibrator 1 can be controlled.

Next, in the case where the variable amplitude section 12 generates a coefficient w(2) and the variable amplitude section 13 generates a coefficient w(3), with respect to a received signal b(t)cos(2π·f1·t) of the vibrator 2, and the received signal of the vibrator 1 also is considered, the output signal of the adding section 15 is represented by the following expression.
Z0(t)≈a(t)cos(2π·f1·t+φa)+b(t)cos(2π·f1·t+φb) (11)

Thus, a phase φb of the received signal b(t)cos(2π·f1·t) of the vibrator 2 also can be controlled, and the received signals of the vibrators 1, 2 in the sub-array 5 can be subjected to phasing addition in the sub-beam former 16. In Expression (11), although the phasing addition by the control of a phase is shown, since there is actually a delay in a received signal owing to the fixed delay section 14, more excellent phasing addition is performed.

Similarly, the received signals of the vibrators 3, 4 in the sub-array 6 can be subjected to phasing addition in the sub-beam former 17. The output signals of the sub-beam formers 16 and 17 are subjected to delay addition in the main beam former 18. Thus, the received signals of the vibrators 1 to 4 in the two-dimensional array 7 are subjected to beam forming.

As described above, according to the ultrasonic diagnostic system of the first embodiment of the present invention, by providing the sub-beam former 16 composed of the amplifying sections 8, 9, the cross point switches 81, 91, the variable amplitude sections 10 to 13, the fixed delay section 14, and the adding section 15, a received signal can be subjected to phasing added with high precision.

### Second Embodiment

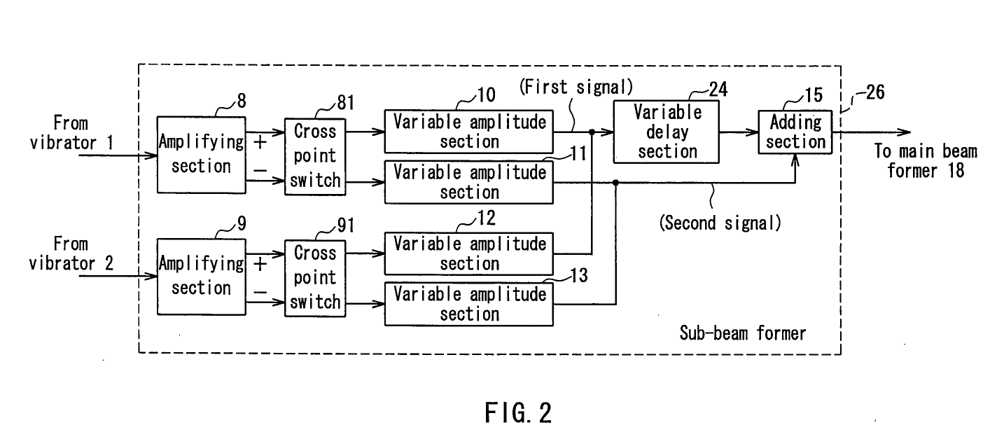

FIG. 2 is a block diagram showing an exemplary internal configuration of a sub-beam former in a receiving section in an ultrasonic diagnostic system according to a second embodiment of the present invention. In the present embodiment, the sub-beam former 16 shown in FIG. 1 referred to in the description of the first embodiment is replaced by a sub-beam former 26 shown in FIG. 2. The remaining configuration is the same as that of the first embodiment.

In FIG. 2, the amplifying sections 8, 9 respectively output a non-inverted output signal (+) and an inverted output signal (−) of received signals. The variable amplitude sections 10, 11 are connected to the amplifying section 8 via the cross point switch 81, and the variable amplitude sections 12, 13 are connected to the amplifying section 9 via the cross point switch 91. The output signals of the variable amplitude sections 10, 12 are added, and the added signal (first signal) is supplied to the variable delay section 24. The output signals of the variable amplitude sections 11, 13 are added, and the added signal (second signal) is added to the output signal of the variable delay section 24 in the adding section 15. The amplifying sections 8, 9, the cross point switches 81, 91, the variable amplitude sections 10, 11, 12, 13, the variable delay section 24, and the adding section 15 constitute the sub-beam former 26.

Next, an operation of the ultrasonic diagnostic system configured as described above will be described.

First, in a fundamental video mode, the frequency of a received signal is f1, the variable delay section 24 imparts a delay time ΔT=T1/4, which is a quarter of one period T1=1/f1 of the received signal, to a signal obtained by adding the signals from the variable amplitude sections 10, 12. In the adding section 15, the received signals of the vibrators 1, 2 are subjected to phasing addition in accordance with Expressions (1) to (11) described in the first embodiment.

Next, in a harmonic video mode, the frequency of a received signal is f2, the variable delay section 24 imparts a delay time ΔT=T2/4, which is a quarter of one period T2=1/f2 of the received signal, to a signal obtained by adding signals from the variable amplitude sections 10, 12. In the adding section 15, the received signals of the vibrators 1, 2 are subjected to phasing addition in accordance with Expressions (1) to (11) described in the first embodiment.

As described above, according to the ultrasonic diagnostic system of the second embodiment of the present invention, by providing the variable delay section 24, the delay time can be varied in accordance with the center frequency of the received signal, and a fundamental video and a harmonic video can be displayed respectively.

### Third Embodiment

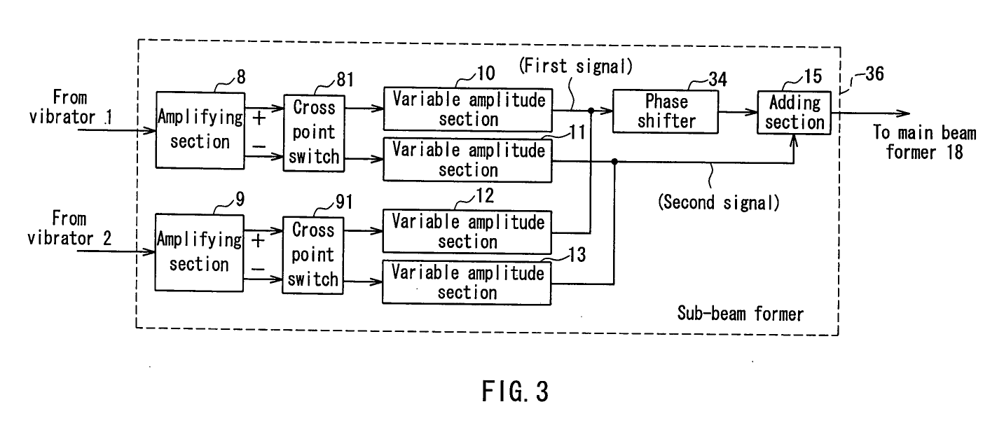

FIG. 3 is a block diagram showing an exemplary internal configuration of a sub-beam former in a receiving section in an ultrasonic diagnostic system according to a third embodiment of the present invention. In the present embodiment, the sub-beam former 16 shown in FIG. 1 referred to in the description of the first embodiment is replaced by a sub-beam former 36 shown in FIG. 3. The remaining configuration is the same as that of the first embodiment.

In FIG. 3, the amplifying sections 8, 9 respectively output a non-inverted output signal (+) and an inverted output signal (−) of received signals. The variable amplitude sections 10, 11 are connected to the amplifying section 8 via the cross point switch 81, and the variable amplitude sections 12, 13 are connected to the amplifying section 9 via the cross point switch 91. The output signals of the variable amplitude sections 10, 12 are added, and the added signal (first signal) is supplied to a phase shifter 34. Furthermore, the output signals of the variable amplitude sections 11, 13 are added, and the added signal (second signal) is added to the output signal of the phase shifter 34 in the adding section 15. The amplifying sections 8, 9, the cross point switches 81, 91, the variable amplitude sections 10, 11, 12, 13, the phase shifter 34, and the adding section 15 constitute a sub-beam former 36.

Next, an operation of the ultrasonic diagnostic system configured as described above will be described.

The frequency of each received signal of the vibrators 1, 2 is f1, and the phase shifter 34 imparts a phase shift to the output signals of the variable amplitude sections 10, 12 so that the phase of each received signal is shifted by 90 degrees (π/2). In the adding section 15, the received signals of the vibrators 1, 2 are subjected to phasing addition in accordance with Expressions (2) to (11) described in the first embodiment.

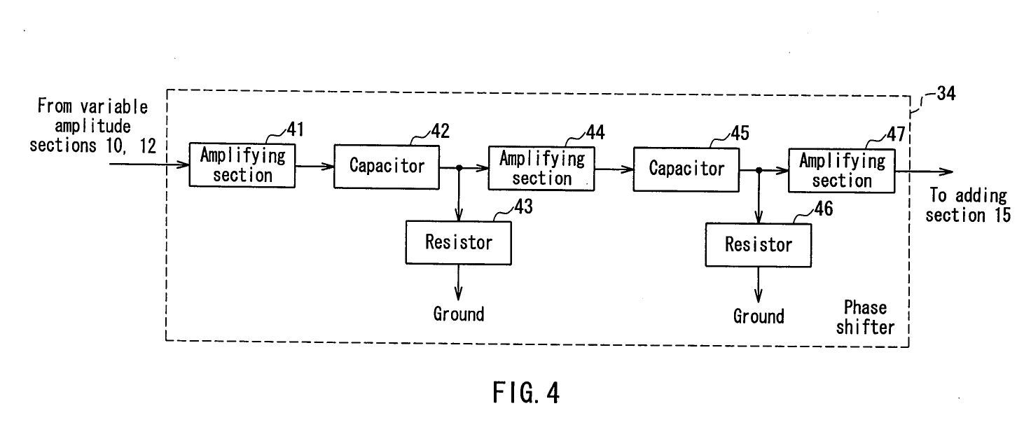

FIG. 4 is a detailed block diagram showing an exemplary internal configuration of the phase shifter 34.

In FIG. 4, the phase shifter 34 is configured so as to have phase shift circuits in two stages, each having a phase shift amount of 45 degrees. Each output signal of the variable amplitude sections 10, 12 is amplified by an amplifying section 41, and has its phase shifted by −45 degrees by the phase shift circuit in the first stage composed of a capacitor 42 and a resistor 43. The signal passing through the phase shift circuit in the first stage is amplified by an amplifying section 44, has its phase shifted by −45 degrees by the phase shift circuit in the second stage composed of a capacitor 45 and a resistor 46, and is amplified by an amplifying section 47 to be output to the adding section 15. Thus, the output signal of the amplifying section 47 has its phase shifted by −90 degrees with respect to the output signal of the amplifying section 41.

As described above, according to the ultrasonic diagnostic system of the third embodiment of the present invention, by providing one phase shifter 34 in each sub-beam former, a received signal can be subjected to phasing addition with high precision. Furthermore, since the phase difference of 90 degrees is realized without using an inductor, the ultrasonic diagnostic system of the third embodiment is advantageous in terms of the miniaturization and the reduction in noise.

### Fourth Embodiment

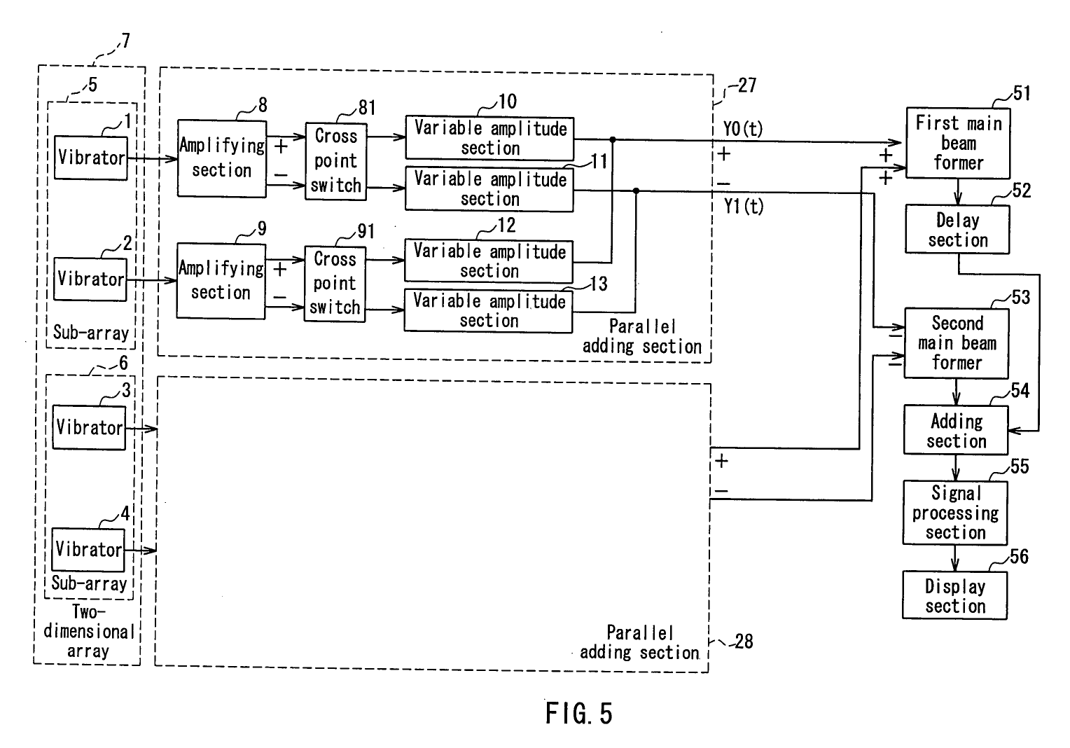

FIG. 5 is a block diagram showing one exemplary configuration of a receiving section in an ultrasonic diagnostic system according to a fourth embodiment of the present invention.

In FIG. 5, vibrators 1 to 4 respectively are composed of electroacoustic transducers, and convert an acoustic echo signal to a received signal. The vibrators 1 and 2 constitute a sub-array 5. The vibrators 3 and 4 constitute a sub-array 6. The sub-arrays 5 and 6 constitute a two-dimensional array 7. The amplifying sections 8, 9 respectively output a non-inverted output signal (+) and an inverted output signal (−) of received signals. Variable amplitude sections 10, 11 are connected to the amplifying section 8 via the cross point switch 81, and the variable amplitude sections 12, 13 are connected to the amplifying section 9 via the cross point switch 91. The output signals of the variable amplitude sections 10, 12 are added to be an added output signal Y0(t) (first signal). The output signals of the variable amplitude sections 11, 13 are added to be an added output signal Y1(t) (second signal). The amplifying sections 8, 9, the cross point switches 81, 91, and the variable amplitude sections 10, 11, 12, 13 constitute a parallel adding section 27.

Furthermore, the received signals from the vibrators 3, 4 are input to a parallel adding section 28. The internal configuration of the parallel adding section 28 is the same as that of the parallel adding section 27.

The non-inverted added output signals of the parallel adding sections 27 and 28 are subjected to delay addition in a first main beam former 51. The inverted added output signals of the parallel adding sections 27 and 28 are subjected to delay addition in a second main beam former 53. The output signal of the first main beam former 51 is delayed in a delay section 52. The output signals of the delay section 52 and the second main beam former 53 are added in an adding section 54, and the output signal of the adding section 54 is processed to be an image signal in a signal processing section 55. The image signal from the signal processing section 55 is displayed on a display section 56.

Next, an operation of the ultrasonic diagnostic system configured as described above will be described.

First, the vibrator 1 generates a received signal a(t)cos(2π·f1·t). Herein, t is a time, a(t) is an envelope of the received signal, and f1 is a center frequency of the received signal. The amplifying section 8 outputs a non-inverted output signal a(t)cos(2π·f1·t) and an inverted output signal −a(t)cos(2π·f1·t). Depending upon the state of the cross point switch 81, the variable amplitude section 10 multiplies the non-inverted output signal or the inverted output signal by a coefficient w(0) to output Y0(t)=±(0)·a(t)cos(2π·f1·t). Depending upon the state of the cross point switch 91, the variable amplitude section 11 multiplies the non-inverted output signal or the inverted output signal by a coefficient w(1) to output Y1(t)=±w(1)·a(t)cos(2π·f1·t).

The same delay time 6 is imparted to the added output signal of the variable amplitude section 10 and the added output signal of the variable amplitude section 11 respectively in the first main beam former 51 and the second main beam former 53. Therefore, in the first main beam former 51 and the second main beam former 53, the phase relationship of the respective outputs Y0(t), Y1(t) does not change.

In the delay section 52, the delay time ΔT=T1/4, which is a quarter of one period T1=1/f1 of a received signal, is imparted to the output signal of the first main beam former 51, so that the output signal Y0(t) has its phase shifted by −π/2 compared with Y1(t). When the output signal of the delay section 52 and the output signal of the second main beam former 53, having such a phase relationship, are added in the adding section 54, as represented by Expressions (3) to (11) described in the first embodiment, the received signals of the vibrators 1, 2 in the sub-array 5 can be subjected to phasing addition. Similarly, the received signals of the vibrators 3, 4 in the sub-array 6 also can be subjected to phasing addition. Thus, the received signals of the vibrators 1 to 4 of the two-dimensional array 7 are subjected to beam forming.

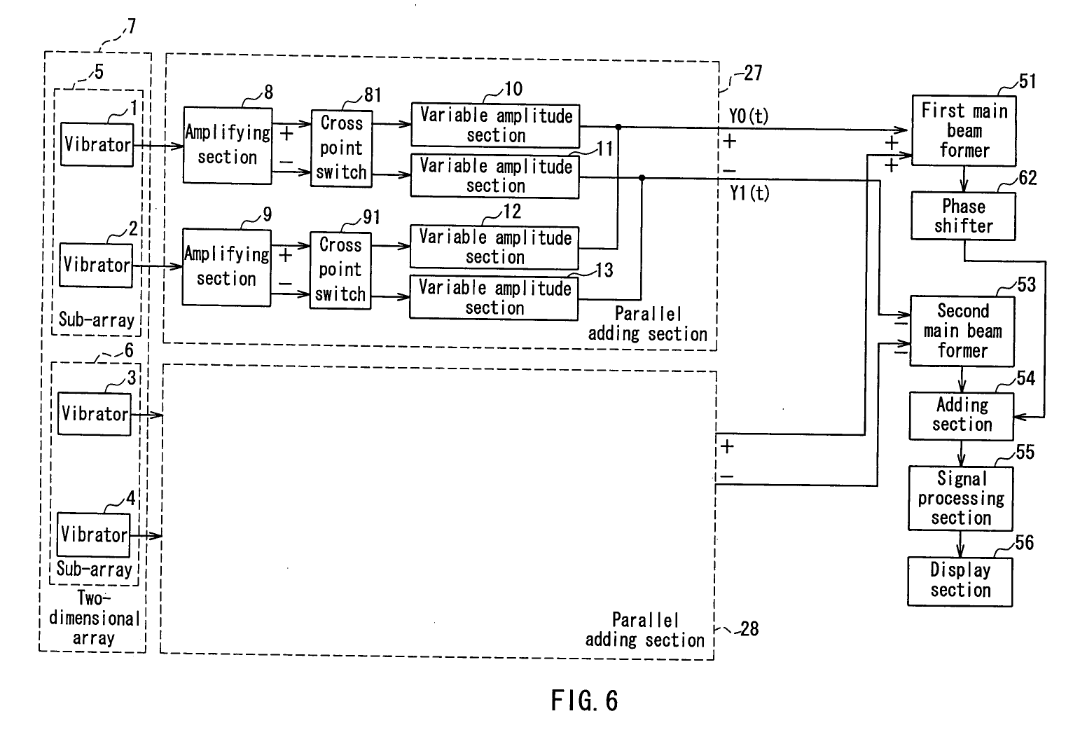

In the above description, an example in which the delay section 52 is provided with respect to the output signal of the first main beam former 51 has been described. However, as shown in FIG. 6, even if a phase shifter 62 is provided with respect to the output signal of the first main beam former 51, the present invention can be carried out similarly.

As described above, according to the ultrasonic diagnostic system of the fourth embodiment of the present invention, by providing the parallel adding sections 27, 28, the first main beam former 51, the second main beam former 53, and the delay section 52, a received signal can be subjected to phasing addition with higher precision.

### INDUSTRIAL APPLICABILITY

The ultrasonic diagnostic system according to the present invention has an advantage that a received signal from an electroacoustic transducer arranged two-dimensionally can be phased with high precision. The ultrasonic diagnostic system according to the present invention is useful as an ultrasonic diagnostic system or the like that has a two-dimensional array and scans a subject three-dimensionally, and can be applied to a medical purpose and the like.

1. An ultrasonic diagnostic system, comprising:
electroacoustic conversion means in which a plurality of sub-arrays, each composed of a plurality of electroacoustic transducers, are arranged at least two-dimensionally;
a sub-beam former that is provided on the sub-array basis, generates signals having different polarities with respect to a received signal from the electroacoustic transducer in the sub-array, obtains a first signal and a second signal by controlling amplitudes of the signals having different polarities of each electroacoustic transducer in the sub-array, followed by adding, imparts a delay time difference corresponding to a quarter of one period of the received signal between the first signal and the second signal by delay means provided inside, and adds the first signal and the second signal to which the delay time difference is imparted; and
a main beam former for subjecting a signal output from the sub-beam former to delay addition.

2. The ultrasonic diagnostic system according to claim 1, wherein the delay means is capable of switching the delay time difference between a quarter of one period of a fundamental of the received signal and a quarter of one period of a harmonic of the received signal.

3. The ultrasonic diagnostic system according to claim 1, wherein the delay means imparts a delay time corresponding to a quarter of one period of the received signal to one of the first signal and the second signal.

4. An ultrasonic diagnostic system, comprising:
electroacoustic conversion means in which a plurality of sub-arrays each composed of a plurality of electroacoustic transducers are arranged at least two-dimensionally;
a sub-beam former that is provided on the sub-array basis, generates signals having different polarities with respect to a received signal from the electroacoustic transducer in the sub-array, and obtains a first signal and a second signal by controlling amplitudes of the signals having different polarities of each electroacoustic transducer in the sub-array, followed by adding, imparts a predetermined phase shift amount to one of the first signal and the second signal by phase shift means provided inside, and adds the first signal or the second signal to which the predetermined phase shift amount is imparted to each other; and
a main beam former for subjecting a signal output from the sub-beam former to delay addition.

5. The ultrasonic diagnostic system according to claim 4, wherein the phase shift means is composed of phase shift circuits in two stages, each having a phase shift amount of 45 degrees, and each of the phase shift circuits in two stages is configured so as to include a capacitor and a resistor.

6. An ultrasonic diagnostic system, comprising:
electroacoustic conversion means in which a plurality of sub-arrays each composed of a plurality of electroacoustic transducers are arranged at least two-dimensionally;
parallel adding means that is provided on the sub-array basis, generates signals having different polarities with respect to a received signal from the electroacoustic transducer in the sub-array, and obtains a first signal and a second signal by controlling amplitudes of the signals having different polarities of each electroacoustic transducer in the sub-array, followed by adding;
a first main beam former for subjecting a first signal added by the parallel adding means to delay addition;
a second main beam former for subjecting a second signal added by the parallel adding means to delay addition;
delay means for imparting a delay time difference corresponding to a quarter of one period of the received signal between an output signal of the first main beam former and an output signal of the second main beam former; and
adding means for adding an output signal of the first main beam former and an output signal of the second main beam former, to which the delay time difference is imparted by the delay means.

7. An ultrasonic diagnostic system, comprising:
electroacoustic conversion means in which a plurality of sub-arrays each composed of a plurality of electroacoustic transducers are arranged at least two-dimensionally;
parallel adding means that is provided on the sub-array basis, generates signals having different polarities with respect to a received signal from the electroacoustic transducer in the sub-array, and obtains a first signal and a second signal by controlling amplitudes of the signals having different polarities of each electroacoustic transducer in the sub-array, followed by adding;
a first main beam former for subjecting a first signal added by the parallel adding means to delay addition;
a second main beam former for subjecting a second signal added by the parallel adding means to delay addition;
phase shift means for imparting a phase difference of 90 degrees between an output signal of the first main beam former and an output signal of the second main beam former; and
adding means for adding an output signal of the first main beam former and an output signal of the second main beam former, to which the phase difference of 90 degrees is imparted by the phase shift means.
